(19) United States
(12) Patent Application Publication
(10) Pub. No.: US 2016/0026894 A1
(43) Pub. Date: Jan. 28, 2016

(54) Ultrasound Computed Tomography

(71) Applicant: Daniel Nagase, Vancouver (CA)

(72) Inventor: Daniel Nagase, Vancouver (CA)

(21) Appl. No.: 14/751,146

(22) Filed: Jun. 26, 2015

Related U.S. Application Data
(60) Provisional application No. 62/029,767, filed on Jul. 28, 2014.

(51) Int. Cl. G06K 9/52; A61B 8/00; A61B 8/15; G06T 5/00; G06T 7/00; G06K 9/46; G06T 11/00; A61B 8/14; A61B 8/08

(52) U.S. Cl. CPC G06K 9/52; A61B 8/14; A61B 8/4245; A61B 8/429; A61B 8/15; A61B 8/4477; A61B 8/5246; G06T 7/0012; G06K 9/46; G06T 11/005; G06T 5/00; G06K 2009/4666; G06T 2207/10132; G06T 2207/10072

(57) ABSTRACT

Ultrasound Computed Tomography is a system of devices that reconstructs a series of 2 dimensional ultrasound images into a 3 dimensional computed tomographic model of an object by cataloguing ultrasound image frames according to the ultrasound probe's position in 3 dimensional space at the time of image capture. Capturing the position and orientation of an ultrasound probe at the time of image frame acquisition allows for appropriate placement of each 2 dimensional ultrasound image within a 3 dimensional computed tomographic workspace. Also subject of this patent is creation of a coherent 3 dimensional model of a body or body part through an algorithmic selection and weighting system to reconcile overlapping regions of ultrasound images captured from different positions. Additionally a system for acquisition of transmission based ultrasound images, made possible by positional cataloguing, is delineated.

### CROSS-REFERENCE TO RELATED APPLICATIONS

Provisional patent No. 62/029,767

### STATEMENT REGARDING FEDERALLY SPONSORED RESEARCH OR DEVELOPMENT (IF APPLICABLE)

Not Applicable

### REFERENCE TO SEQUENCE LISTING, A TABLE, OR A COMPUTER PROGRAM LISTING COMPACT DISC APPENDIX (IF APPLICABLE)

Not Applicable

### BACKGROUND OF THE INVENTION

Ultrasound imaging has become a staple within the area of medical imaging. Its advantages include the ability to capture real time images of various body parts without any radiation exposure. Basic ultrasound imaging involves transmission of ultrasound waves through a transducer into body tissues. The reflected ultrasound waves are then picked up by the ultrasound transducer, and plotted according to location of reflection for the x axis, and time of reflection for the y axis, in order to generate a 2 dimensional crossectional image of the body part being imaged. Many structures within the body can be well visualized with ultrasound imaging, however, it has its limitations. All ultrasound imaging is highly dependent on the conductivity of the body's tissues at ultrasound frequencies. High water content tissues are the best conductors of ultrasound, while air, bone and adipose tissue are poor conductors. When ultrasound waves encounter poor tissue conductors of sound, ultrasound images distant to the poorly conductive tissue are difficult or impossible to attain. For this reason, multiple repositioning maneuvers are used by the ultrasound operator manipulating the ultrasound probe to visualize around the ultrasound attenuating tissue.

When used for diagnostic purposes, multiple images of a body part or organ are taken in order to get an overall picture of that area. The images are then later reviewed by a radiologist and a medical report is given. While an ultrasound operator may take images of a body part from different perspectives, current “3d ultrasound” images are limited by the fact that all the 2 dimensional image frames used to create a “3d” image, are acquired from only one ultrasound probe location on the body part being examined. This is due to an inability to cross reference images taken from different locations on the body part with respect to each other, as the position of the ultrasound probe during an image acquisition session is highly variable.

While standardized positions do exist for obtaining ultrasound images from various body parts, the actual ultrasound image viewed during a session with a particular patient is highly dependent on the patient's individual body shape, and the ultrasound operator's technique during that session on that day. Unlike the case with x-ray computed tomography commonly known as a CT scan, where x-ray beams are passed through the body part from multiple known locations, ultrasound imaging by nature uses image acquisition from points in space that vary from person to person, and session to session. This limits the ability to make a complete 3 dimensional tomographic reconstruction a body part, especially one that is viewable from different angles and across different cross sections that were not captured initially during an ultrasound session.

To optimize ultrasound images the following positioning techniques are used by the operator of the ultrasound probe (also known as transducer) to avoid ultrasound attenuating tissue that can act as a barrier to detailed ultrasound imaging.

1) Rotation of the probe around its longitudinal axis

2) Horizontal motion across the body part

3) Vertical motion across the body part

4) Angling around the axis of the ultrasound and body interface. (changing the angle of the ultrasound probe's contact with the body part.)

5) Pressure (Pushing into the body part to displace poorly conductive tissue)

These cardinal motions are illustrated in FIG. 1:

In addition to the spatial dimensions of repositioning and rotation, the 5^{th} axis that an ultrasound operator can use to obtain a clearer ultrasound image is pressure. By pressing the ultrasound probe more firmly against a compliant body part such as the abdomen, the operator can sometimes obtain an improved image from the ultrasound probe. In the case of the abdomen, increased pressure on the probe can displace ultrasound attenuating tissue such as fat or gas bubbles within superficial structures, to allow for better transmission and reflection of ultrasound waves from deeper body structures within the abdomen such as the kidneys.

Rationale:

Whereas ultrasound probe repositioning and pressure can achieve an adequate image for a number of medical diagnoses, operator and patient factors such as body habitus (obese vs. skinny) can preclude the acquisition of high quality images necessary for investigation of other medical conditions. Furthermore, while images of structures that are superficial or close to the ultrasound transducer can be extremely detailed, to the millimeter resolution, deeper structures cannot be seen in as much detail due to frequency limitations and attenuation of the reflected ultrasound waves bounced back from deeper body organs. Ultrasound computed tomography allows for the interpolation of multiple ultrasound images taken from multiple varied locations, and their 3 dimensional reconstruction into a coherent 3 dimensional topographic model of the body part.

### SUMMARY OF TERMS

Session: An instance where a patient's body occupies a position in space for examination by an ultrasound probe.

3 Dimensional Orientation Device: A marking unit attached to a device or body part that allows that object's position and orientation in space to be acquired.

Ultrasound Probe: Device to transmit ultrasound pulses and capture ultrasound reflections from a body part. The probe then transmits this information to a computer that renders the ultrasound reflections into a 2 dimensional grayscale image.

Probe-body interface: The point in space where the ultrasound probe contacts the body part being examined.

Ultrasound Image Frame: A 2 dimensional grayscale image created from ultrasound reflections recorded by an ultrasound probe. Each point in the 2 dimensional grayscale image is calculated by the position of the ultrasound reflection on the head of the ultrasound probe and time of reflection. When the position of the ultrasound reflection is plotted on the X axis, and the time on the Y axis, a 2 dimensional image can be generated by the computer the ultrasound probe is connected to. As deeper reflections take a longer time to reach the ultrasound probe head, plotting the time of reflection on the Y axis equates the Y axis with depth of the ultrasound reflecting structure. The intensity of reflection is encoded by the shade of grayscale, where higher intensity reflections are encoded by brighter pixels.

Ultrasound Probe Pressure: pressure exerted at the interface between the ultrasound probe and the body part being examined.

### BRIEF SUMMARY OF THE INVENTION

1) A positional cataloguing system for the ultrasound probe:
a. While the ultrasound probe is acquiring images from the body part being examined, a spatial cataloguing system records the probe's instantaneous 3 dimensional positions and orientations in space during the session.
b. The 3 dimensional positions and orientations in space are recorded over the time that the probe is acquiring images from the patient. Each 3 dimensional position and orientation coordinate for the ultrasound probe is associated to the ultrasound image frame recorded at that corresponding point in time.

2) A positional cataloguing system for the body part being examined:
a. While the ultrasound probe is acquiring images from the body part being examined, a spatial cataloguing system records any variations of that body part's 3 dimensional position in space.
b. The 3 dimensional position in space of the body part is recorded over the time that the probe is operational and acquiring images. Each 3 dimensional coordinate for body position at any point in time is associated to the ultrasound image frame taken from the ultrasound probe at that corresponding point in time.

3) A pressure cataloguing system for the ultrasound probe:
a. While the ultrasound probe is acquiring images from the body part being examined, a pressure cataloguing system records the probe's physical pressure against the body at the probe-body interface.
b. The pressure of the probe against the probe-body interface is associated to each ultrasound image frame and the ultrasound probe's 3 dimensional position in space at that moment in time.

4) 3 dimensional Computed tomographic modeling of the body part being imaged:
a. Compilation of data containing the spatial coordinates and orientation of the ultrasound probe together with the spatial coordinates and orientation of the body part being examined. Parsing of this metadata into each ultrasound image frame recorded during a session.
b. Calculation of the ultrasound probe's relative position with respect to the body part via subtraction of the body part's 3 dimensional position in space from the ultrasound probe's 3 dimensional position in space.
c. Assignment of a position, in a computed 3 dimensional workspace, of each 2 dimensional ultrasound image frame, based on the ultrasound probe's known orientation and position in space. Because each 2 dimensional ultrasound image frame occupies a fixed spatial relationship to the ultrasound probe's head, a 3 dimensional view of the body part being imaged by the ultrasound probe can be created by ordering each cross sectional 2 dimensional ultrasound image frame adjacent to its neighboring image frames, derived from the ultrasound probe's physical position in space. The spatial relationship of each frame to other frames is calculated from the spatial orientation and position coordinates of the ultrasound probe recorded in the metadata of each ultrasound image frame.
d. Reconciliation of overlapping areas of ultrasound images using an adaptive algorithm that adjusts for differing image quality between frames and within frames.
e. 3 dimensional Computer Rendering and Reconstruction of the body part being imaged into a 3 dimensional computed tomographic model of the body part. This computed reconstruction can be reviewed after an ultrasound session has ended, and can be re-rendered to provide additional 2 dimensional ultrasound images from views not physically possible. I.e. With a virtual ultrasound probe placed within the abdomen, a view of a body part from within that body part can be created.
   i. Each ultrasound image frame acquired during a session, cross referenced to it's position within the body part being imaged, creates a 3 dimensional computed tomographic model of that body part, that can be reverse rendered after a session has been completed, to provide 2 dimensional images from different perspectives not originally acquired during the session.

5) Two probe Technique:
a. A system of acquiring additional ultrasound images via ultrasound conductance through a body part instead of the currently established standard of generating images from ultrasound reflections from structures within the body part.
b. Two identical ultrasound probes similar to the ultrasound probe used and described in parts 1-3 above are placed approximately across from each other, each alternately transmitting ultrasound pulses to the other probe and receiving ultrasound signals from the other probe through a body part.
c. When operating in a dual probe mode, the ultrasound imaging computer coordinates each probe such that each alternates between sending and receiving ultrasound waves through the body part. When one probe is transmitting ultrasound pulses, the other is coordinated to receive and vice versa.
d. Overlapping areas of image frames are reconciled as described in part 4d.

6) Pressure based Computed tomographic modeling of the body part being imaged.
a. Compilation of pressure data at the probe-body interface with the spatial orientation data of the ultrasound probe described above.
b. Referenced to the pressure at the probe-body interface, a series of 3 dimensional Computer rendered tomographic reconstructions of a body part are ordered along a scale of ultrasound probe pressure at the body contact area.
   i. At various degrees of deformation of the body part, as measured by a combination of pressure at the probe-body interface and probe position in space, a separate 3 dimensional Computed Tomographic reconstruction is made of said body part.
   ii. Each of these 3 dimensional Computed Tomographic reconstructions are arranged such that they can be reviewed as a series. Changes of the body part with variations in ultrasound probe pressure can then be visualized by viewing this pressure based series of 3 dimensional reconstructions as a scrollable series through time.

### BRIEF DESCRIPTION OF THE SEVERAL VIEWS OF THE DRAWING

FIG. 1: Cardinal motions used in the operation of an ultrasound probe.

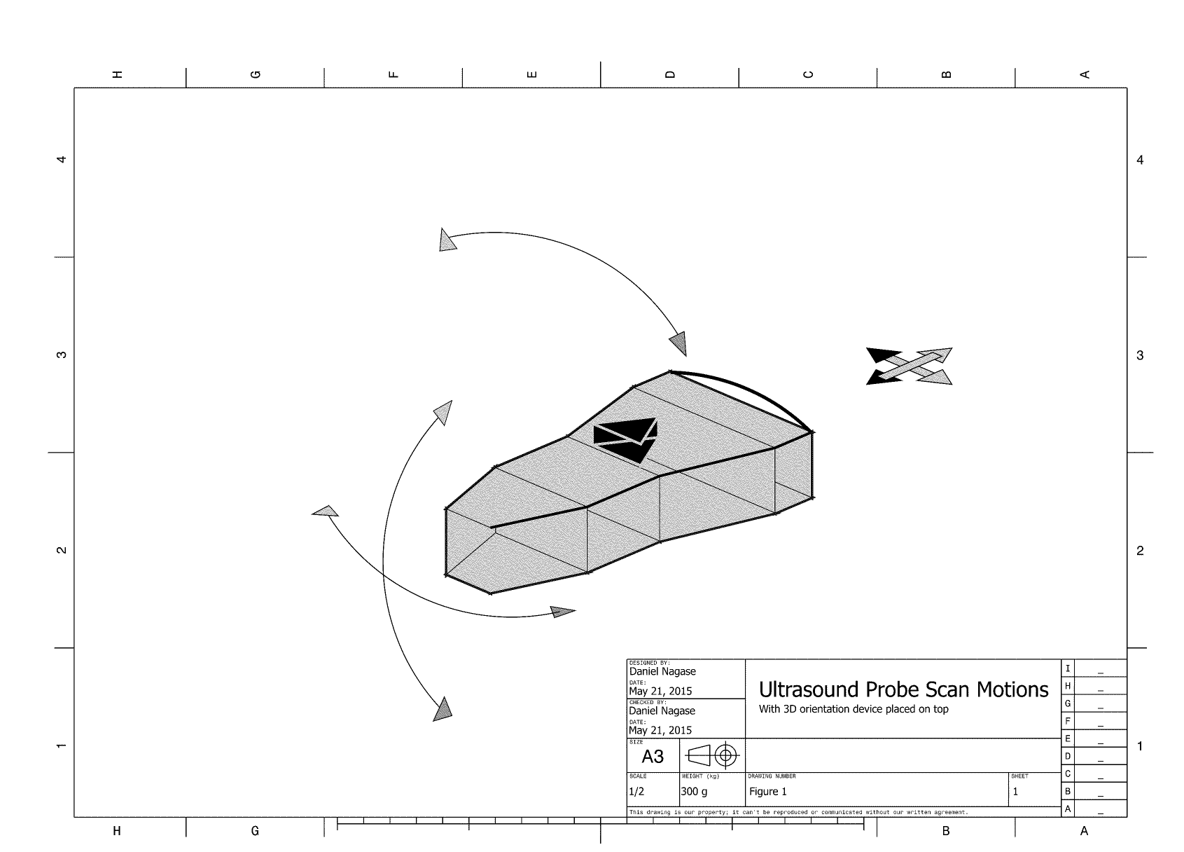

FIG. 2: Overall layout of an ultrasound computed tomography system.

FIG. 3: 3 dimensional spatial orientation device.

FIG. 4: Measurement of signal to noise ratio from the standard deviation of surrounding pixels.

FIG. 5: Measurement of signal to noise ratio from the standard deviation of pixels over time.

FIG. 6: Vector based weighting of data pixels generated by ultrasound reflections.

FIG. 7: Mechanism by which diffraction gain augments ultrasound energy available for reflections when attenuation based weighting along a vector is calculated.

### DETAILED DESCRIPTION OF THE INVENTION

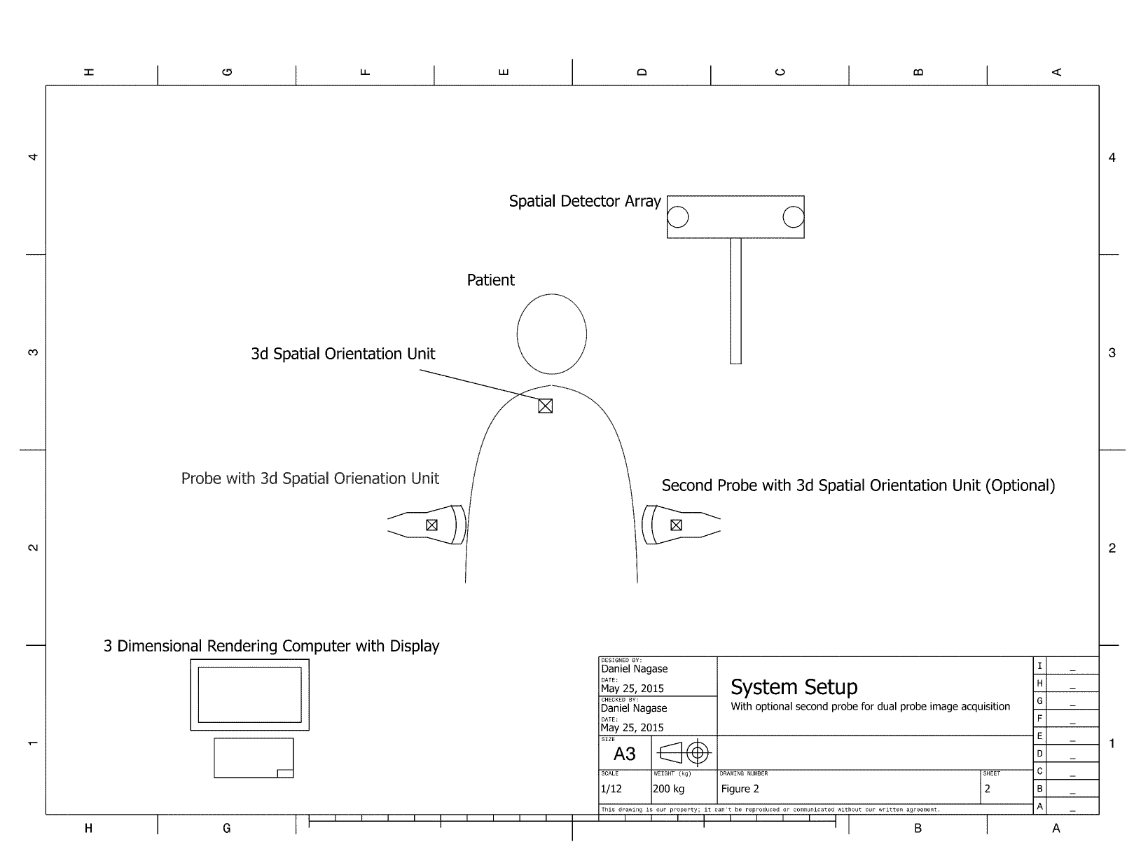

Please refer to FIG. 2, for the overall layout of an ultrasound computed tomography system.

1) A positional cataloguing system for the ultrasound probe:

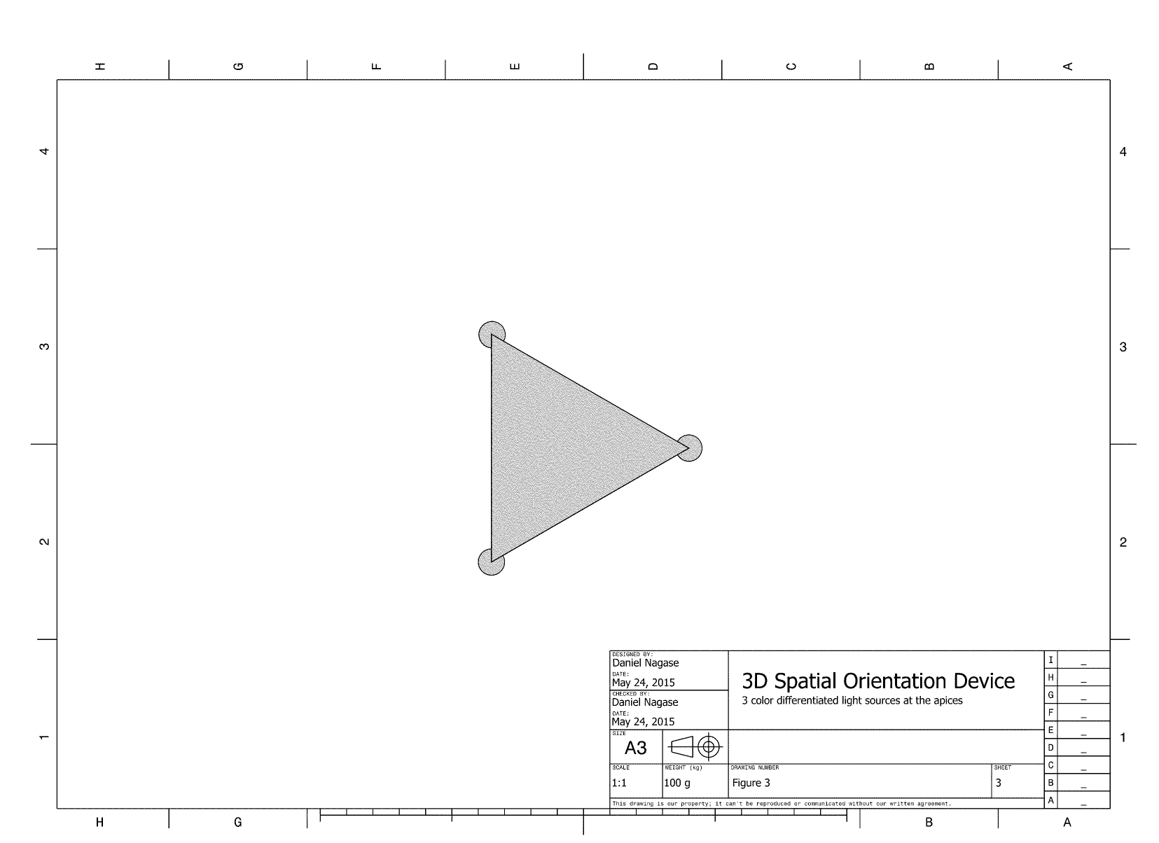

The spatial cataloguing system to record an ultrasound probe's 3 dimensional position in space consists of 2 components:
a) 3 dimensional spatial orientation unit. (FIG. 3)
   i. The unit consists of 3 light emitting elements each of a different colour arranged at the apices of an equilateral triangle. The light emitting elements may be colored LED's, or a single LED with 3 colored optic branches.
   ii. This unit is affixed to the ultrasound probe.
b) Spatial detector array.
   i. A stereoscopic digital video device consisting of two video cameras a set distance apart capable of recording the position of the 3 dimensional spatial orientation unit located on the ultrasound probe from 2 different perspectives.
   ii. The spatial detector array is located above the patient and ultrasound operator such that its view of the 3 dimensional spatial orientation unit on the ultrasound probe is unhindered during operation of the ultrasound probe. This elevated location can be attained by mounting the spatial detector array on the ceiling or a sufficiently tall tripod.

This cataloguing system allows for recording and measurement of minute variations in the position of the ultrasound probe over time via parallax between the two digital video cameras. This cataloguing system also allows for recording and measurement of minute variations in the orientation of the ultrasound probe over time via the videographic measurement of the relative positions of each light emitting element with respect to the other two light emitting elements on the spatial orientation device.

2) A positional cataloguing system for the body part being examined:

The spatial cataloguing system to record the position of the patient's body part in 3 dimensional space consists of 2 components:
a) 3 dimensional spatial orientation unit.
   i. The unit consists of 3 light emitting elements each of a different colour, arranged at the apices of an equilateral triangle. The light emitting elements may be colored LED's, or a single LED with 3 colored optic branches.
   ii. This unit is placed on the patient in a location adjacent to but not overlapping the area to be examined.
   iii. The color of the 3 light emitting elements on the patient's 3 dimensional spatial orientation unit are different from that of the 3 dimensional spatial orientation unit on the ultrasound probe.
b) Spatial detector array.
   i. A stereoscopic digital video device consisting of two video cameras a set distance apart, capable of recording the position of the 3 dimensional spatial orientation unit located on the patient from 2 different perspectives.

This cataloguing system allows for recording and measurement of minute variations in the position and orientation of the patient over time via parallax between the two digital video cameras, and videographic measurement of the relative position of each light emitting element with respect to the other two light emitting elements on the spatial orientation device placed on the patient.

3) A pressure cataloguing system, for the ultrasound probe:

The pressure cataloguing system acquires real time data through a thin film pressure sensor integrated into the ultrasound probe head. This data is recorded with each ultrasound image frame along with the ultrasound probe's 3 dimensional position in space.

4) 3 dimensional Computed modeling of the body part being imaged:

Each ultrasound image frame is placed within a computed 3 dimensional workspace according to its location, as calculated by the position and orientation of the ultrasound probe subtracted from the position and orientation of the patient, as recorded by the digital videographic spatial detector array, at the time of image frame capture. The fixed spatial relationship between an ultrasound image and the ultrasound probe allows placement of each 2 dimensional ultrasound image frame within a 3 dimensional digital workspace based on the ultrasound probe's physical location and orientation.

The following are abbreviations for the 3 dimensional coordinates and orientations in space representing the relevant variables for calculating each ultrasound image frame's placement within the 3 dimensional computed tomography model:

1) Pp=3 dimensional position and orientation of the ultrasound probe.

2) Bp=3 dimensional position and orientation of the patient's body part.

3) Ip=3 dimensional Image to Probe spatial constant

4) Pi=image position and orientation of a 2 dimensional ultrasound image frame within the 3 dimensional computed tomographic model

Ip represents the 3 dimensional constant describing the positional relationship between the 3 dimensional spatial orientation unit affixed to the ultrasound probe, and the 2 dimensional ultrasound image frame generated by said probe.

The equation to resolve the position of each 2 dimensional ultrasound image frame within a computed 3 dimensional space, is as follows:

Pi=(Pp−Bp)+Ip

During a session, the spatial detector array makes a stereoscopic digital video recording of each of the 3 dimensional spatial orientation units located on the ultrasound probe and patient's body part respectively. Parallax between the two digital video recordings allows for the calculation of the distance and position of each of the 3 dimensional spatial orientation units relative to the spatial detector array.

The stereoscopic spatial detector array also determines the orientation and angle of the ultrasound probe to the patient along with any changes to orientation and angle over time. The relative positions of the 3 light emitting elements within each 3 dimensional spatial orientation unit is used to calculate the angle and orientation of the probe and patient during a session. As the ultrasound probe is rotated or angled, each light emitting element on the 3 dimensional spatial orientation unit will move either closer to or further away from the other two light emitting elements. Since each light emitting element occupies a fixed and known physical relationship to the other two light emitting elements on the face of an equilateral triangle, the angle and orientation of the ultrasound probe can be calculated from the relative distances of each light emitting element from the other two. Differing the colors of each light emitting element on the spatial orientation unit aids in the speedy recognition of the ultrasound probe's orientation by the spatial detector array.

The ultrasound probe's position and orientation in space, as calculated from the parallax between the 2 digital videographic recordings of the probe's 3 dimensional spatial orientation unit, is subtracted from the parallax derived position and orientation of the patient's 3 dimensional spatial orientation unit, and added to the 3 dimensional Image to Probe constant to yield the relative position and orientation of each 2 dimensional ultrasound image frame within the computed tomographic model of the body part being examined. The coordinates of position and orientation for both the patient and ultrasound probe are parsed into the metadata of each ultrasound image frame.

The position of a 2 dimensional ultrasound image frame within 3 dimensional space, as generated by subtracting the position and orientation coordinates of the ultrasound probe from the position and orientation coordinates of the body part being imaged, plus the image to ultrasound probe positional constant, applied cumulatively over multiple 2 dimensional ultrasound image frames taken from multiple positions and locations on a body part, yields a 3 dimensional reconstruction of the body part being imaged. However, before a 3 dimensional reconstruction from multiple 2 dimensional images placed in a 3 dimensional digital workspace can be rendered into a coherent 3 dimensional computed topographic model, areas where 2 dimensional images overlap must be reconciled.

5) System for adaptive reconciliation of 2 dimensional data from overlapping ultrasound image frames:

A rudimentary 3 dimensional model composed of multiple 2 dimensional ultrasound image frames in its raw form requires algorithmic image processing before a useful 3 dimensional reconstruction can be created for diagnostic review. Whenever multiple 2 dimensional ultrasound image frames overlap in a 3 dimensional computed tomographic workspace, a system of image selection, reconciliation and averaging must be employed to create the most coherent composite from a series of 2 dimensional image frames with variable areas of overlap.

To produce a coherent 3 dimensional tomographic model as described in part 4) 3 dimensional Computed modeling of the body part being imaged, areas for which there are multiple ultrasound images of a given position in 3 dimensional space must be reconciled. The overlapping 2 dimensional image data must be processed to maximize the contribution of high signal and low noise images to the overall tomographic 3 dimensional model, and minimize the contribution of poor quality ultrasound image data to areas where there already exists high quality and useful data. Without a system of image reconciliation, the 3 dimensional tomographic model described in part 4) 3 dimensional Computed modeling of the body part being imaged would be unintelligible and or degraded if poor quality ultrasound images are collected during a session.

First, poor quality images, or image frames captured while the ultrasound probe is not contacting the patient must be discarded.
1) When Ultrasound probe pressure, as measured by a thin film pressure sensor integrated into the ultrasound probe head falls below a certain operator set threshold, indicating that the probe is either not contacting the patient, or being moved from one position to another, that ultrasound image frame's contribution to the overall 3 dimensional reconstruction is set to 0.
2) When an image frame captured from the ultrasound probe has an overall contrast to noise ratio below a certain operator set threshold, that corresponding image frame's contribution to the overall 3 dimensional computed tomographic reconstruction is set to 0.

For the remaining images where there exists spatial overlap within the 3 dimensional computed tomographic reconstruction, a mathematical function based weighted averaging algorithm is employed to reconcile the multiple data points occupying the same position in 3 dimensional space, in order to provide the clearest image possible when there exists one or more areas of overlap across multiple 2 dimensional image frames.

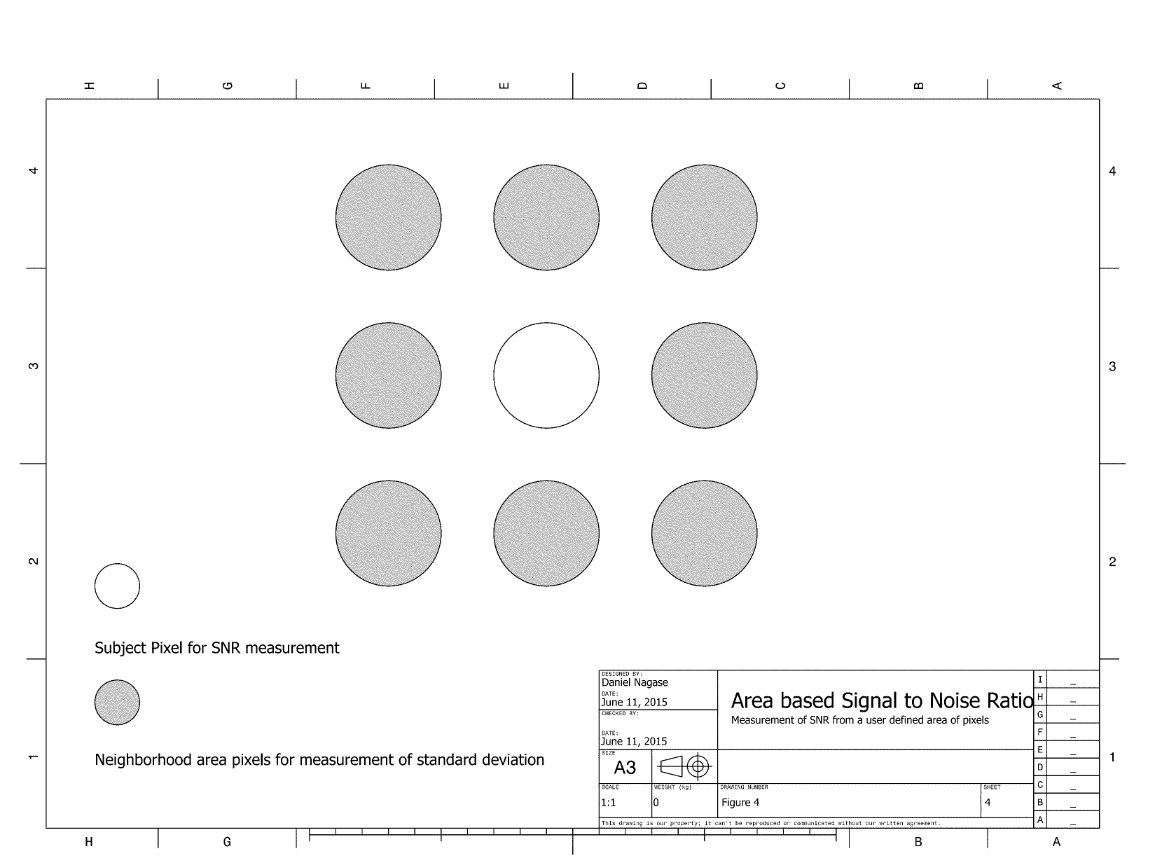

Signal to noise ratio (hitherto abbreviated SNR) based weighting of image overlap areas can take 2 forms. If overlap areas exist from images taken at different ultrasound probe positions as recorded by the spatial orientation device, SNR measurement is performed on each overlap area within a frame on an area basis, by comparing the expected value of a pixel's intensity to the standard deviation of pixel intensities in a user pre-defined neighborhood area of adjacent pixels within an ultrasound image frame. (FIG. 4)

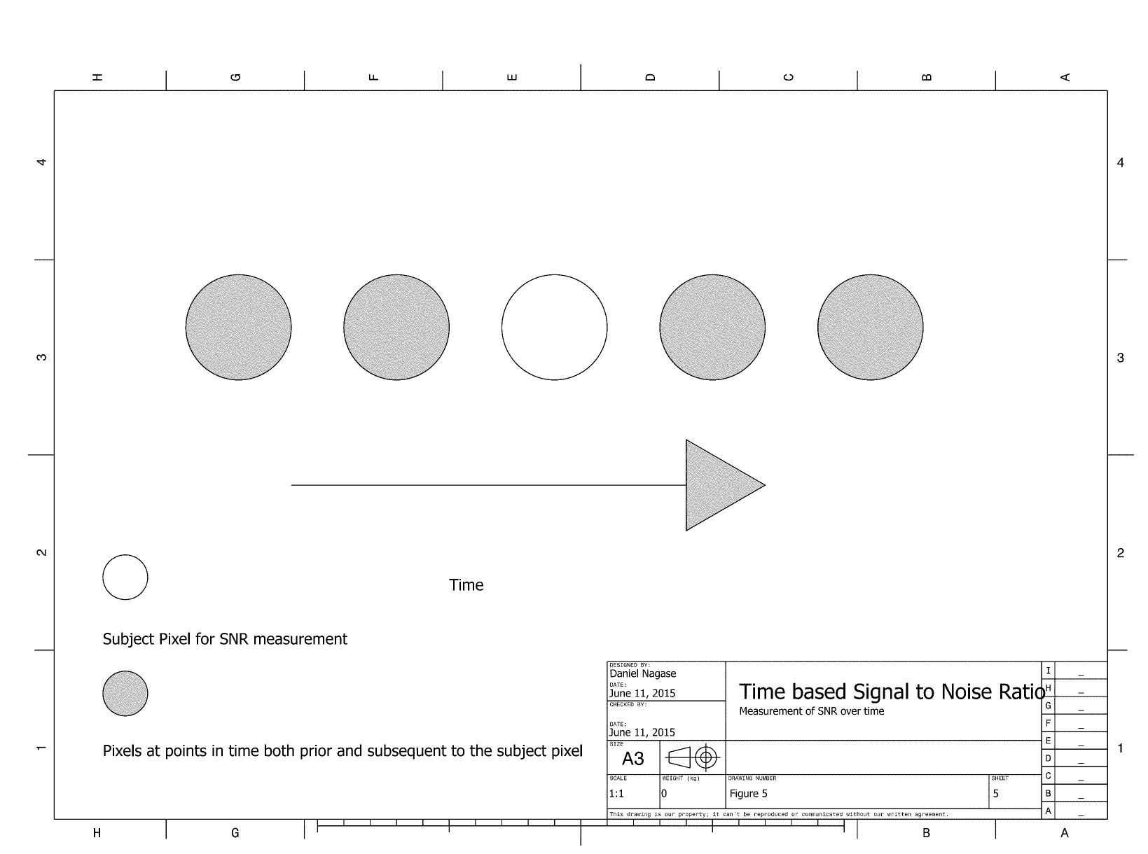

If multiple image frames exist from one spatial probe position as measured by the spatial orientation device, then SNR can also be measured by comparison of a pixel to other pixels occupying that same point in 3 dimensional space but at adjacent points in time both prior to and subsequent to the point in time in question. (FIG. 5)

One or both methods of signal to noise ratio measurement may be employed for the purposes of assigning a SNR to each pixel of ultrasound data. Subsequently, an operator selectable curve of pixel weighting per signal to noise ratio can be used to optimize an image according to overall ultrasound conditions. For example, to maximize the relative contribution of a pixel with a high signal to noise ratio to the final value of a point in space where multiple overlapping pixels exist, an exponential relationship between SNR and weighting may be employed. On the other hand, a linear curve may be selected by the ultrasound operator if a body part is particularly difficult to image clearly, and multiple low signal to noise ratio images need to be combined for a clear overall image. Irrespective of the exact mathematical function to assign a weight to each level of SNR, the weighted average of all the overlapping pixels at a point in 3 dimensional space will then form the final value of the pixel occupying that corresponding point in space within the 3 dimensional computed tomographic reconstruction.

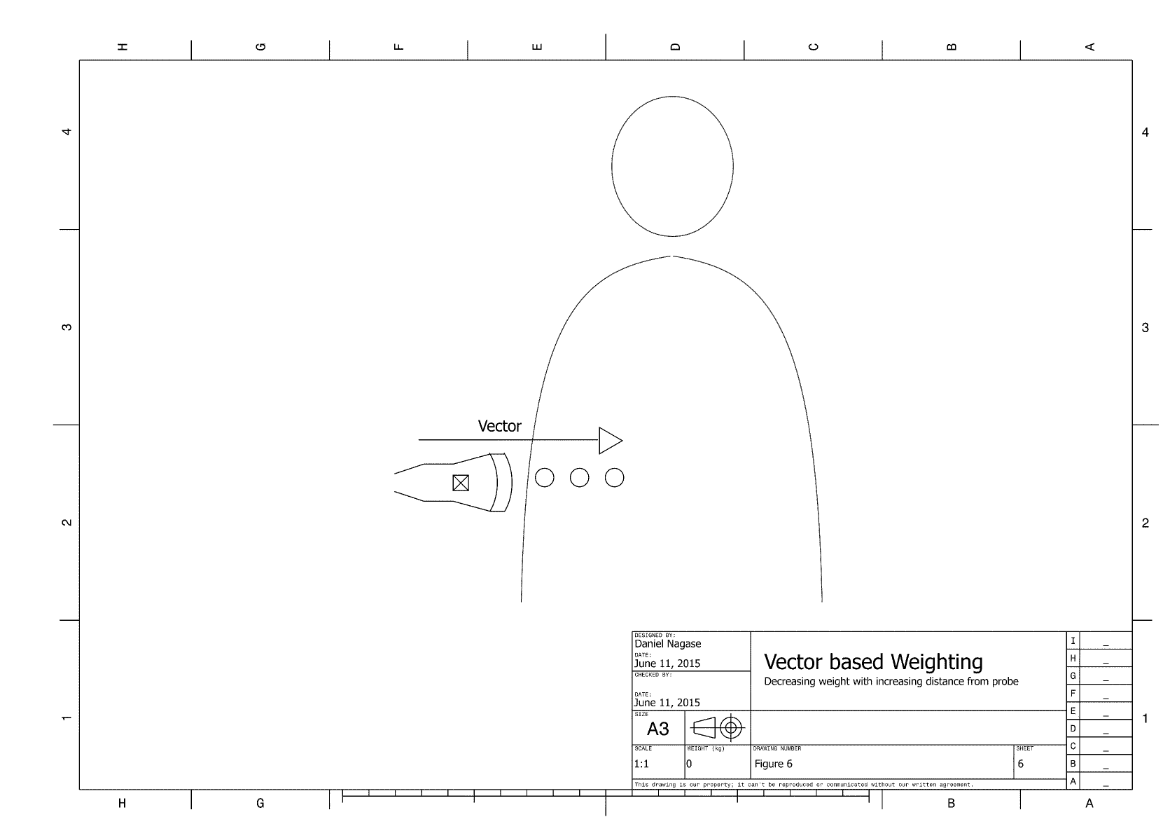

A second weighting system that can be used as in conjunction with SNR weighted averaging is vector based weighted averaging. For a vector based weighted average, a vector is first assigned to each pixel based on the forward axis of the ultrasound probe at time of image pixel capture. (FIG. 6) Dependent on the time of reflection, a distance from the ultrasound probe is assigned to each pixel. Pixels placed more proximally along an ultrasound probe's vector receive a higher weighting than pixels placed more distant along a vector. Thus when pixels overlap in situations where the ultrasound probe captures images of an area from two different positions, the pixels captured when the ultrasound probe is close by (i.e. the pixel is quite proximal along an ultrasound probe's vector) are weighted higher than the pixels captured when they are more distant along the ultrasound probe's vector, i.e. pixels captured when the ultrasound probe is far away. This weighting system accounts for the phenomenon of acoustic attenuation, where ultrasound waves lose intensity as they progress deeper into a body part, thus making their reflections progressively weaker in comparison to background noise, as the distance from the ultrasound probe increases. The exact mathematical function to assign a weight to each pixel can be user selectable dependent on the expected acoustic attenuation properties of the body part being examined and the frequency of ultrasound being used.

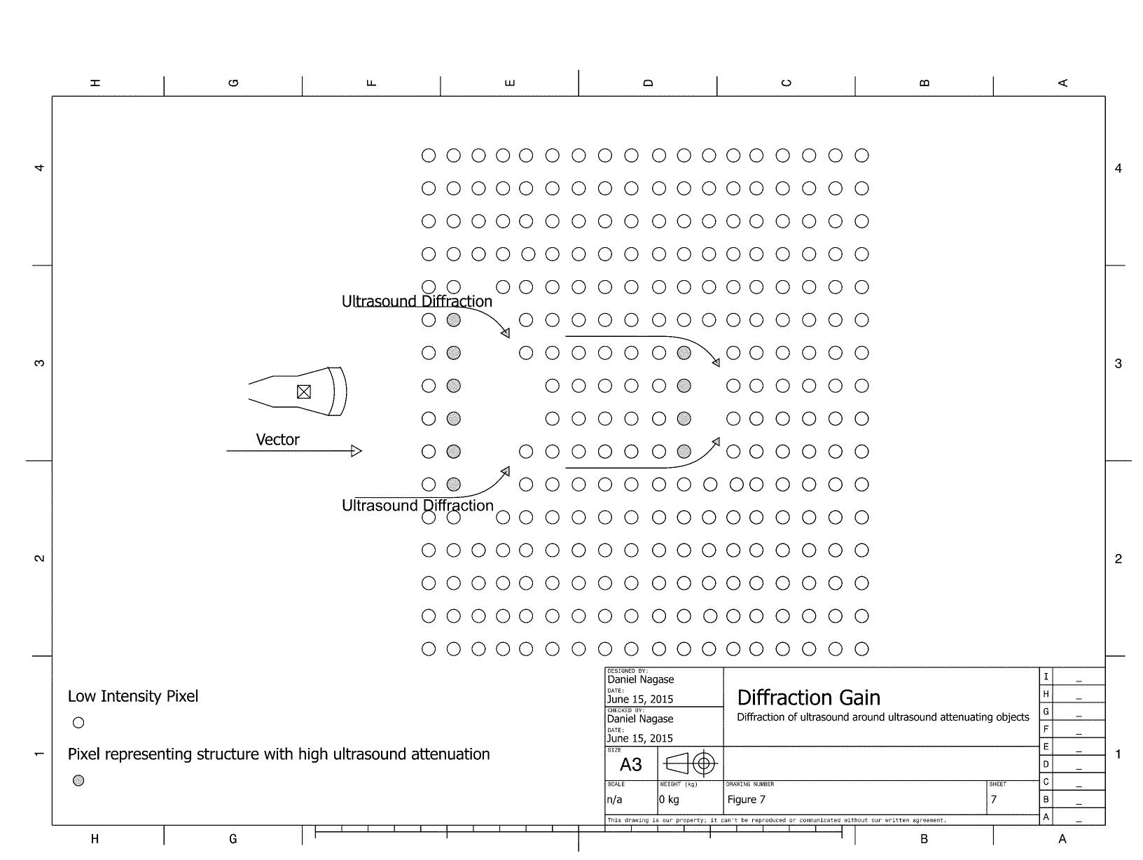

A second methodological implementation of vector based weighting that can be used alone or in conjunction with the proximity based weighting along a vector as described above, is attenuation based vector weighting calculated from the actual attenuation values of each pixel on every pixel distal to it along an ultrasound vector. Conceptually this is best understood through an example of a highly dense and ultrasound reflective object within a body part. Once an ultrasound wave hits that object within a body part, it generates a strong reflection which is visualized as a group of pixels of high intensity. Pixels distal to the high intensity pixels along an ultrasound vector will have little to no ultrasound signal to reflect due to the presence of an acoustic shadow behind that highly attenuating object. However, at a certain distance behind that ultrasound attenuating object, especially when it is small, ultrasound waves are again able to generate reflections due to ultrasound diffraction around the attenuating object. (FIG. 7) This effect is called diffraction gain, and is a confounding variable that must be adjusted for when using vector based weighting as a function of attenuation values instead of distance.

As the intensity of a pixel represents its reflectivity to ultrasound, a pixel's intensity can be used to predict the remaining ultrasound energy distal to it along its vector. To calculate an attenuation based vector weight for a pixel, first, an attenuation value is assigned to every pixel present proximally along the ultrasound vector from which that pixel was captured. This attenuation value is calculated as a mathematical function of the ultrasound frequency and the intensity of all the pixels preceding a subject pixel along its vector. A pixel with high intensity assigns a high attenuation value for all points distant to it along its vector, as that pixel represents a point in space that reflects most of the ultrasound energy sent to it from the ultrasound probe. Points distant along that same ultrasound vector have their weight reduced by an attenuation value derived as a mathematical function of all the individual intensities of all the pixels present proximally along said vector.

However, ultrasound waves do not only travel in straight lines. As a function of frequency, ultrasound can diffract around the edges of an object with high ultrasound reflectivity. (FIG. 7) Thus depending on the size of the ultrasound reflective object, and distance behind that object, ultrasound energy reappears, increasing with distance behind the ultrasound reflective object, and increasing with decreasing size of said object. This phenomenon shall be hitherto referred to as “diffraction gain”. This effect acts in an opposing manner to acoustic attenuation of ultrasound energy.

While attenuation weighting adjusts for the decrease in ultrasound energy along a vector, creation of a meaningful weighting system for the purposes of generating an accurate vector attenuation weighted image requires that diffraction gain be included in the weighting of each pixel. This is needed to adjust for the augmentation of ultrasound energy with increasing distance from an ultrasound attenuating structure. Diffraction gain along a pixel's vector increases with decreasing size of an ultrasound attenuating object and increasing distance from said object.

The diffraction gain is calculated as a mathematical function of the attenuation values of a user pre-defined and variable 3 dimensional area proximal to a pixel in question. Calculating diffraction gain first requires the assignment of attenuation values to pixels in the 3 dimensional region both surrounding and proximal to a pixel in question. This is performed in the manner previously described. (i.e. A mathematical function of the ultrasound frequency and reflection intensities of the series of pixels directly proximal to each pixel along each pixel's ultrasound vector.) If attenuation values are high in the user pre-defined 3 dimensional neighborhood of pixels proximal to a pixel of interest, the amount of diffraction gain for pixels immediately distal to the point in question is low.

However, if attenuation values in a 3 dimensional neighborhood of surrounding pixels proximal to a particular pixel are low, then even if the pixel series directly proximal to that pixel in question have high attenuation values, (i.e. presence of high intensity pixels directly preceding the subject pixel along its vector) there can be significant diffraction gain, allowing structures behind that pixel to generate a significant and meaningful ultrasound reflection. These pixels receive a mathematically derived augmentation to their vector attenuation based weighting in the form of diffraction gain.

The area and shape of the neighborhood pixels over which diffraction gain is calculated is user pre-defined and variable. Factors influencing the size and shape of the sampling area include but are not limited to, ultrasound frequency, tissue density, and presence of known acoustically shadowing structures within an area of examination.

(FIG. 7)

The vector based attenuation weight (Wt) applied to a pixel (p) is as follows:

Wt=C(A+Df) i)

Wt=the vector based attenuation weight of a pixel

C=constant for ultrasound probe type and frequency being used

A=attenuation value as calculated from pixel intensity of preceding pixels.

Df=diffraction gain

x=the distance from the ultrasound probe head

ii) The attenuation value A for pixel (P), is calculated as a function (F) of the intensity (I_{1,2,3} . . . ) of each pixel proximal to pixel (P) along its vector, divided by function (f) of (x,_{1,2,3} . . . ) where x represents the distance of each pixel preceding pixel P from pixel P.

A=[F(I₁)/f(x₁)]+[F(I₂)/f(x₂)]+[F(I₃)/f(x₃)] . . .

iii) The diffraction gain (Df) for each pixel P is calculated by mathematical function G applied to the attenuation values (A_{a,b,c} . . . ) of which a,b,c, . . . represent the pixels within an operator pre-defined 3 dimensional area proximal to pixel P along its vector.

Df=G(A_{a,b,c} . . . )

Attenuation vector based weighting resolves ultrasound data around structures that create sonic shadowing, and when applied to a system of weighted averaging of pixels captured from multiple different vectors, it can provide an accurate reconciliation of overlapping data in difficult ultrasound imaging situations, such as imaging around bones.

With real time 3 dimensional modeling, the system for reconciliation adapts to new ultrasound image frames as they are acquired. i.e. As new 2 dimensional images overlap with old ones, continuous recalculation of the weighted average of the overlapping areas improves the 3 dimensional model iteratively during a session. Real time iterative weighting and reconstruction during a session can alert an operator to areas that may need additional images taken for completion of an accurate 3 dimensional computed tomographic model of a body part.

Other proprietary and non-proprietary image processing algorithms may be used in addition to SNR weighted averaging, vector based weighted averaging as a function of distance, and vector attenuation based weighted averaging. Optimization of each 2 dimensional ultrasound image with other digital image processing algorithms may be prior to image overlap reconciliation, subsequent to it, or both pre and post the aforementioned overlap reconciliation and 3 dimensional reconstruction.

6) Dual probe based image acquisition:

With 2 ultrasound probes, a second mode of image acquisition is available through transmitted ultrasound. Via ultrasound conductance through a body part, ultrasound images are generated by sending pulses of ultrasound energy from a sending probe, whose position and orientation in space are known via the methods described in section 1) A positional cataloguing system for the ultrasound probe, to a receiving probe whose position and orientation in 3 dimensional space are also known through the same positional cataloguing process detailed in section 1) A positional cataloguing system for the ultrasound probe.

Two identical ultrasound probes are utilized in a dual probe system, each capable of both transmitting ultrasound pulses and receiving both transmitted and reflected ultrasound energy. When operating in a dual probe mode, the ultrasound imaging computer coordinates each probe such that each alternates between sending and receiving ultrasound pulses through the body part. When one probe is transmitting ultrasound pulses, the other is receiving and vice versa.

Each two dimensional transmitted ultrasound image frame is placed in a 3 dimensional computed workspace as described in section 4) 3 dimensional Computed modeling of the body part being imaged. This digital workspace is separate from, but capable of being overlaid upon the previously described 3 dimensional computed topographic model created from ultrasound reflection data.

Overlapping areas of image frames are reconciled as described in part 5) System for adaptive reconciliation of 2 dimensional data from overlapping ultrasound image frames.

While each probe is in receiving mode, the ultrasound probe can simultaneously collect the reflected ultrasound from it's own transmission pulse while at the same time collecting transmitted ultrasound data from the other probe's transmission pulse. The receiving mode for each probe is synchronized to ultrasound pulse transmission from the other probe. This allows collection of transmitted ultrasound images at the same time as reflected ultrasound images.

To differentiate signals from ultrasound reflections with imaging data from ultrasound transmission, a system of alternating frequencies is used where each probe transmits at a frequency different from that of the other probe. i.e. Ultrasound probe A transmits ultrasound pulses at frequency X, while Ultrasound probe B transmits ultrasound pulses at frequency Y. Ultrasound probe A, immediately after transmitting a pulse at frequency X, reverts to ultrasound reception mode to receive ultrasound reflection data from its own pulse at frequency X. Concurrently, Ultrasound Probe B transmits an ultrasound pulse at frequency Y. Ultrasound Probe A then receives this transmission ultrasound signal at frequency Y and differentiates it from its own ultrasound reflection signal at frequency X generated by its own pulse. From the perspective of Probe B, immediately after sending a pulse at Frequency Y, Probe B reverts to reception mode, and receives reflected ultrasound signals at frequency Y while at the same time receiving transmitted ultrasound signals from Probe A at frequency X.

The ultrasound imaging computer coordinates the timing of each probe's transmission pulse and reception mode according to the speed of ultrasound through the body part being examined and the distance between the two probes as measured through the methods described in part 1) A positional cataloguing system for the ultrasound probe.

For diagnostic imaging review, the transmission based 3 dimensional ultrasound tomographic model can be overlaid upon the reflection based ultrasound 3 dimensional tomographic reconstruction with a user variable transparency. This facilitates comparison views of structures within a body part according to their ultrasound reflectivity, transmissibility or a combination of both.

7) Pressure based Computed tomographic modeling of the body part being imaged

Ultrasound head pressure as measured by a thin film pressure sensor on the ultrasound probe head is recorded during a session. Each 2 dimensional ultrasound image frame is then grouped into a user pre-defined number of groups corresponding to a user pre-defined range of ultrasound probe head pressures. Separate 3 dimensional computed tomographic ultrasound image reconstructions are then made by the methods described in parts 1 through 5 above, for each grouping of ultrasound head pressures. Each 3 dimensional reconstruction is ordered such that it can be scrolled through both forwards and backwards along a 4^{th} dimension, i.e. time, such that it provides a “movie” view of how the ultrasound image of a body part changes with pressure.

As ultrasound probe pressure changes during acquisition of images from a location on the patient's body, the image quality of each ultrasound image frame changes with pressure. Superficial structures are better seen at lighter pressures while clearer imaging of deeper structures may be facilitated by increased ultrasound head pressures. Furthermore, the compliance of a body part and the structures within it, can be measured by correlating the change in position of the ultrasound probe with the pressure at the ultrasound probe head. Compliance, and deformation of structures with pressure provides additional medical diagnostic information on the body part being examined.

1) System for cataloguing the relative orientation and position of an ultrasound probe with respect to a body part in 3 dimensional space.

2) System for cataloguing the pressure applied by an ultrasound probe to a body part.

3) System for creation of a 3 dimensional tomographic model of a body part from multiple 2 dimensional ultrasound image frames spatially assigned to positions in a digital 3 dimensional workspace based on the real world 3 dimensional position of the ultrasound probe capturing those images at the time of image capture.
(To calculate the position of each 2 dimensional ultrasound image frame within a 3 dimensional space for the purposes of 3 dimensional tomographic modeling, the physical position of the ultrasound probe relative to the body part must be known for every frame of 2 dimensional ultrasound image capture. claim 3 is thus dependent on claim 1 and claim 2, and is the logical utility and employment for collection of such data as described in claim 1 and claim 2.)

4) System for adaptive reconciliation of data from overlapping areas of 2 dimensional ultrasound image frames.
i) Data reconciliation in the form of mathematical function based, weighted averaging of overlapping regions across multiple ultrasound image frames.
ii) A final value for a pixel or pixel group in 3 dimensional space equaling the weighted average of all the pixels or pixel groups within the area of overlap where regions of multiple 2 dimensional ultrasound image frames occupy the same area in 3 dimensional space.
iii) Combination of multiple data reconciliation methods for areas of ultrasound image frames that occupy the same 3 dimensional position in space. The types of data reconciliation methods are described in claim 5, claim 6, claim 7, claim 8, claim 9, claim 10, and claim 11.

5) Method to apply signal to noise ratio based weighting of areas of overlap within ultrasound image frames for the purposes of weighted averaging of said areas of overlap as described in claim 4.
i) A user adjustable parameter to apply signal to noise ratio weighting to individual pixels, or variably sized pixel groups within each 2 dimensional ultrasound image frame.
ii) An operator adjustable set of mathematical functions to assign a weight to each pixel or pixel group within an ultrasound image frame using that pixel or pixel group's signal to noise ratio compared to the best signal to noise ratio of a corresponding pixel or pixel group within the area of overlapping ultrasound image data.

6) Continuous updating of the best signal to noise ratio within areas of overlap performed in real time throughout the course of an ultrasound session.

7) A user selectable and combinable method for calculation of signal to noise ratio within ultrasound image frames using area based signal to noise ratio measurement; time based signal to noise ratio measurement; or a combination of both.

8) When area based signal to noise ratio measurement is employed for claim 5, claim 6 and claim 7, a method for the user to define the neighborhood area of pixels within ultrasound image frames over which signal to noise ratio is measured.
Prior art—area based signal to noise measurement calculates the signal to noise ratio of a pixel by its standard deviation from the pixel's expected value, as a function of the standard deviation of the pixel values in a user defined neighborhood of surrounding pixels.

9. time based signal to noise ratio measurement is employed for claim 5, claim 6 and claim 7, a method for the user to select the amount of time over which signal to noise ratio is measured when consecutive ultrasound image frames are taken from a single spatial location as determined by claim #1.
Prior art—on a pixel by pixel basis, time based measurement of the signal to noise ratio is performed by comparison of a pixel's value to the standard deviation of pixel values for other pixels occupying that same point in space, but at adjacent points in time both prior to and subsequent to the point in time when the pixel in question was acquired.

10) A system for assigning a vector based weight to each pixel in an ultrasound image frame based on the distance from the ultrasound probe head—for the purposes of weighted averaging as described in claim 4.
A mathematical function to assign weights to each pixel in an ultrasound image frame based on the spatial vector from which the pixel was captured and the distance from the ultrasound probe head as measured by the apparatus in claim 1. This weight assigned to each pixel is then used in the calculation of a vector weighted average for all pixels overlapping at a point in 3 dimensional space.

11) A system for assigning a vector attenuation based weight to each pixel in an ultrasound image frame based on the amount of ultrasound energy present at the physical location represented by said pixel—for the purposes of weighted averaging as described in claim #4.
i) System for estimating the amount of ultrasound energy present at a pixel by calculating the attenuation of ultrasound energy prior to a pixel as a function of ultrasound frequency and the ultrasound attenuation of structures preceding a pixel along its vector.
ii) Method to measure ultrasound attenuation as a mathematical function of the intensities of a series of pixels preceding a point in space along a vector derived from an ultrasound probe's position and orientation.
iii) Method to correct for augmentation of ultrasound energy at a point in space arising from diffraction.
iv) Estimation of ultrasound energy augmentation at a point in space arising from diffraction, as a mathematical function of the attenuation values of pixels in a 3 dimensional region preceding a pixel along its vector.
v) Application of an operator adjustable set of variables for size and shape of the 3 dimensional region over which diffraction augmentation of ultrasound energy for a point in space is calculated.

12) System to coordinate and produce a composite weighted average for every point in 3 dimensional space where one or more ultrasound image frames have regions of overlap using the different methods described in claim 5, claim 6, claim 7, claim 8, claim 9, claim 10, and claim 11.
i.e. Implementation of weighted averaging based on vector, weighted averaging based on signal to noise ratio, weighted averaging via vector attenuation or a combination of all 3.

13) Application of an operator adjustable value for a poor signal to noise ratio or whole frame contrast to noise ratio, below which image frames or sections of image frames are automatically discarded.

14) System for creation of a series of 3 dimensional tomographic models of a body part as referenced in claim 3, consecutively arranged by pressure of the ultrasound probe head at the probe to body part interface.

15) Application of an operator adjustable threshold for low ultrasound probe head pressure below which ultrasound image frames are automatically discarded.

16) A dual probe method for capturing ultrasound images via ultrasound energy transmission through a body part.

17. using the dual probe method as described in claim 16, a method for differentiation of transmitted and reflected ultrasound signals by frequency.

18. using the dual probe method as described in claim 16, a computer based system for synchronization between each probe for ultrasound pulse transmission and reception such that each probe alternately receives ultrasound signals while the other probe transmits and vice versa.

19) A system where each ultrasound probe while in receiving mode, collects the reflected ultrasound signal from it's own transmission pulse concurrently with the transmitted signal from the other probe's transmission pulse.
This claim allows collection of transmitted ultrasound images at the same time as reflected ultrasound images.

20) Generation of an ultrasound image from computed modeling of ultrasound signals transmitted through a body part.
i) Imaging of structures within a body part by calculation of absorption of ultrasound ultrasound energy between a transmitting and receiving probe whose positions and orientations in space are known via the methods described in claim 1.
ii) Image placement of this transmission based 2 dimensional ultrasound image within a 3 dimensional workspace based on the 3 dimensional positions of the transmitting and receiving probes via methods as described in claim 3.
iii) Image processing and overlap reconciliation for transmitted ultrasound image frames via methods as described in claim 5, claim 6, claim 7, claim 8, claim 9, claim 10, and claim 11.

21) System to allow overlap and/or overlay of transmission based ultrasound images with reflection based ultrasound images within a 3 dimensional computed tomographic reconstruction of a body with variable transparency.
A system to allow for variable transparency of transmission based 3 dimensional computed tomographic ultrasound reconstructions, when overlayed with reflected ultrasound 3 dimensional computed tomographic reconstructions.
